(19) United States
(12) Patent Application Publication
(10) Pub. No.: US 2011/0106029 A1
(43) Pub. Date: May 5, 2011

(54) POLYP TRAP

(76) Inventors: Mary L. Garren, Carmel, CA (US); Lloyd R. Garren, Carmel, CA (US); Silas Lum, Monterey, CA (US); Daniel G. Luba, Monterey, CA (US); James A. Disario, Carmel, CA (US); Mark Vierra, Monterey, CA (US)

(21) Appl. No.: 12/917,761

(22) Filed: Nov. 2, 2010

Related U.S. Application Data
(60) Provisional application No. 61/257,200, filed on Nov. 2, 2009.

(51) Int. Cl. A61M 1/00

(52) U.S. Cl. 604/319

(57) ABSTRACT

A polyp trap comprises a body portion and a top portion sealingly and releasably covering the body portion. A suction entrance passageway in the top portion receives liquid and tissue during examination procedures. A removable tissue collection basket in the body portion has a bottom wall with small drainage openings therein, and a removable screen is positioned in the body portion below the collection basket. A suction discharge passageway in the body portion is located below the removable screen, and the discharge passageway is connected to a suction source for the passage of liquid from the body portion.

### CROSS REFERENCE TO RELATED APPLICATIONS

The present application claims the benefit of provisional application Ser. No. 61/257,200, filed Nov. 2, 2010, for all useful purposes, and the specification and drawings thereof are included herein by reference.

### FIELD OF THE INVENTION

The present invention relates to a tissue trap for use in capturing tissue samples during endoscopic and other related medical procedures.

### BACKGROUND OF THE INVENTION

Endoscopic procedures are frequently performed for both diagnostic and therapeutic reasons. These procedures often result in the production of tissue that needs to be collected and sent for further analysis. The current ways of retrieving samples of cut tissues consists of mechanical nets, forceps, snares and suction traps. This suction trap is connected to the endoscopic suction port and allows tissue and fluid to flow through the device inlet and traps the tissue samples before reaching the suction outlet. The currently available devices are expensive and often retain fluid or leak when opened which can contaminate work areas and personnel. There remains a need for a polyp trap, which does not hold residual fluid or leak, and one in which it is easy to see tissue, and which facilitates retrieval of multiple specimens.

Polyp traps and the like are disclosed in U.S. Pat. No. 4,643,197 granted Feb. 17, 1987, US 2006/0189950 published Aug. 24, 2006, and US 2007/0191731 published Aug. 16, 2007, all of which are incorporated herein by reference.

### SUMMARY OF THE INVENTION

The present invention is a polyp trap that has an inlet tube from the endoscopic suction port which is centered over a removable basket which sits in the lip of the suction device. The basket has an easy grasp bar and the basket sits over a secondary screen to further insure no loss of tissue samples. This screen sits directly over the suction outlet. The trap is shaped to minimize residual fluid retention and not leak when opened. It has a second screen to minimize any tissue loss. The tray and screen are white to help facilitate tissue recovery by color contrast. The seal of the trap is formed by suction approximating the lid in the lip of the body of the trap, and the trap is designed to open with easy finger pressure on the lid flange handle. There is also a stop to prevent over opening, which could contaminate adjacent working surfaces. The inner suction tube provides a direction of the suctioned material and allows the material to empty into the trap when opened while preventing spillage of material still in the suction line. The bottom suction tube sticks up into the trap forming a reservoir so there is no contamination of work surfaces.

### BRIEF DESCRIPTION OF THE DRAWINGS

Novel features and advantages of the present invention in addition to those noted above will be become apparent to persons of ordinary skill in the art from a reading of the following detailed description in conjunction with the accompanying drawings wherein similar reference characters refer to similar parts and in which:

FIG. 1 is an exploded perspective view of a polyp trap, according to the present invention;

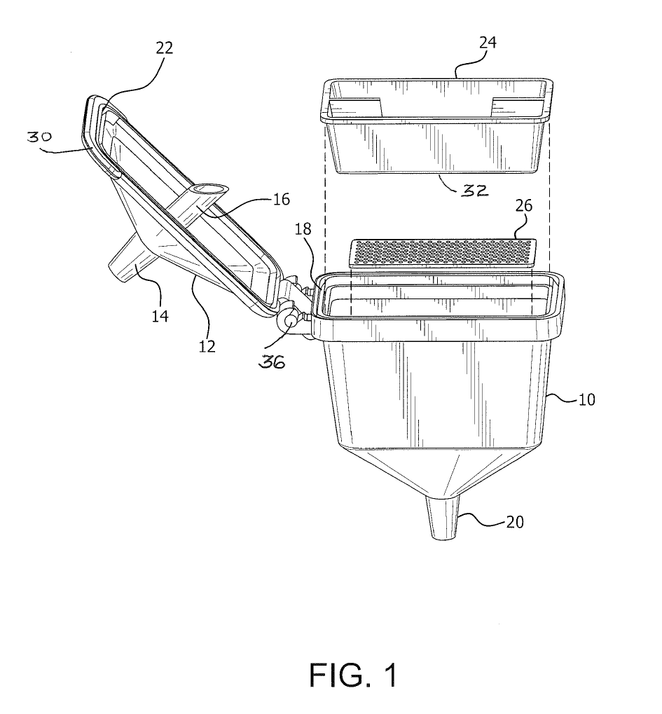

FIG. 2 is a front elevational view with portions in section of the polyp trap shown in FIG. 1;

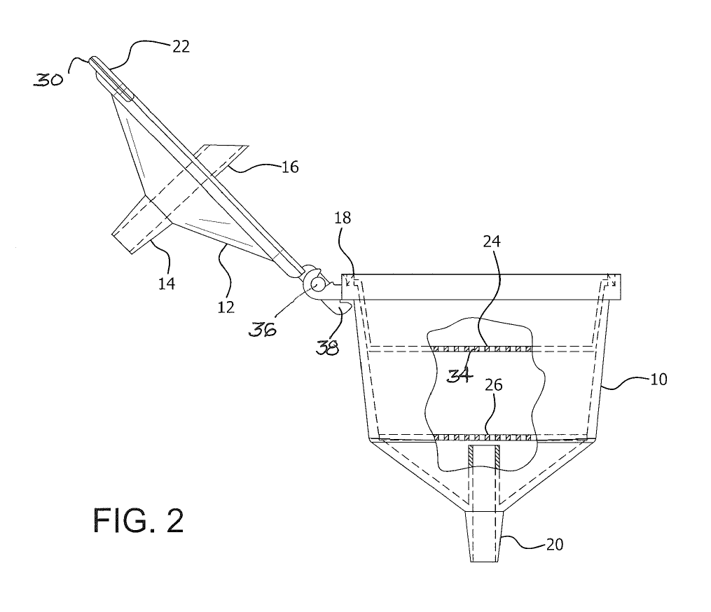

FIG. 3 is an exploded front elevational view of the polyp trap shown in FIGS. 1 and 2;

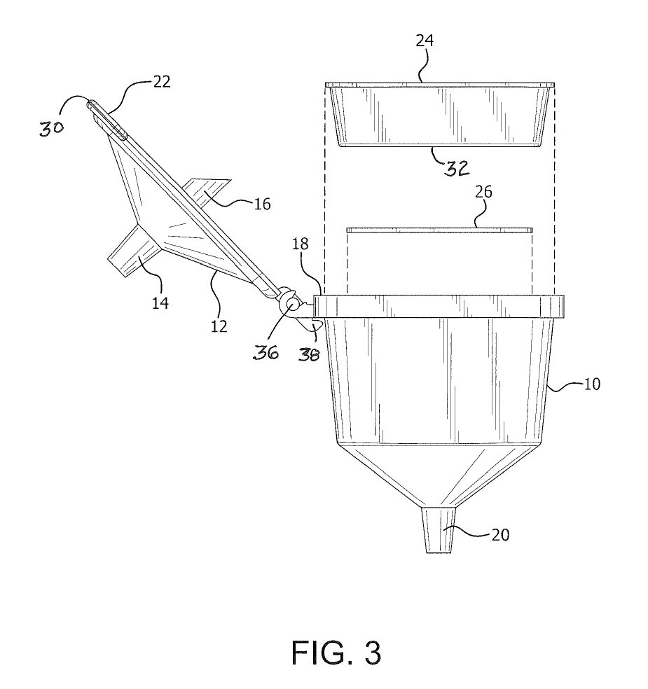

FIG. 4 is another exploded perspective view of the polyp trap shown in FIGS. 1-3;

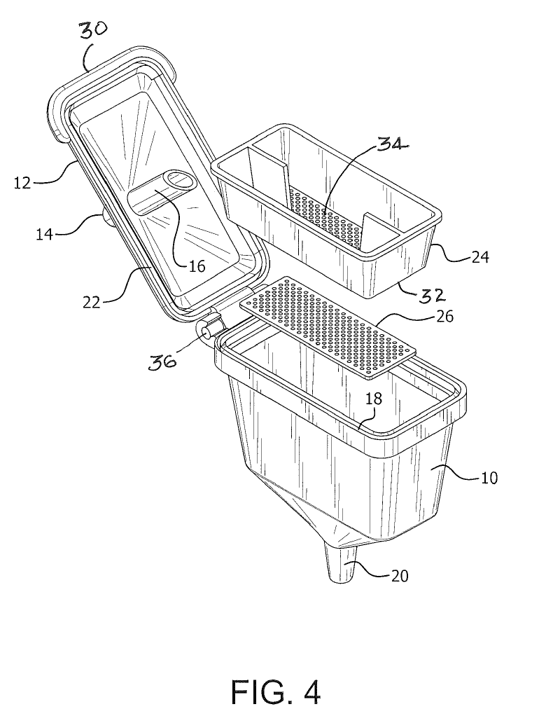

FIG. 5 is a front elevational view of the polyp trap shown in FIGS. 1-4; and

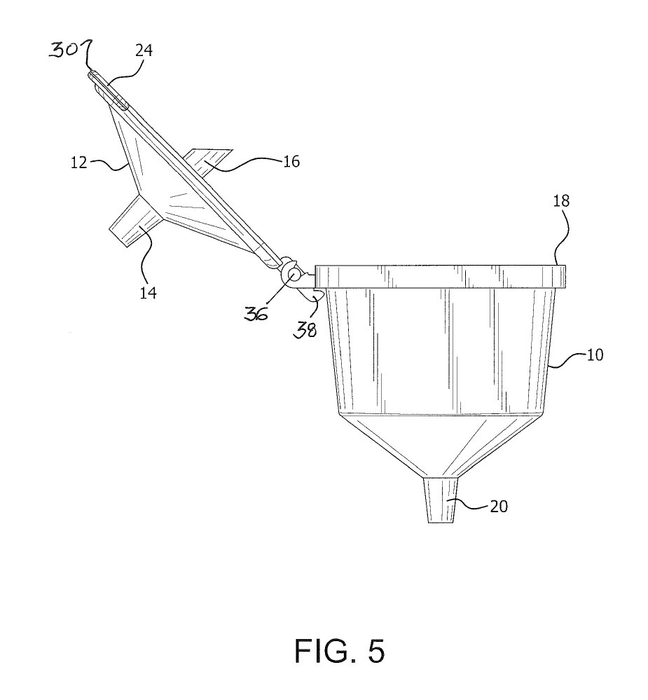

FIG. 6 is a perspective view of the polyp trap shown in FIGS. 1-5.

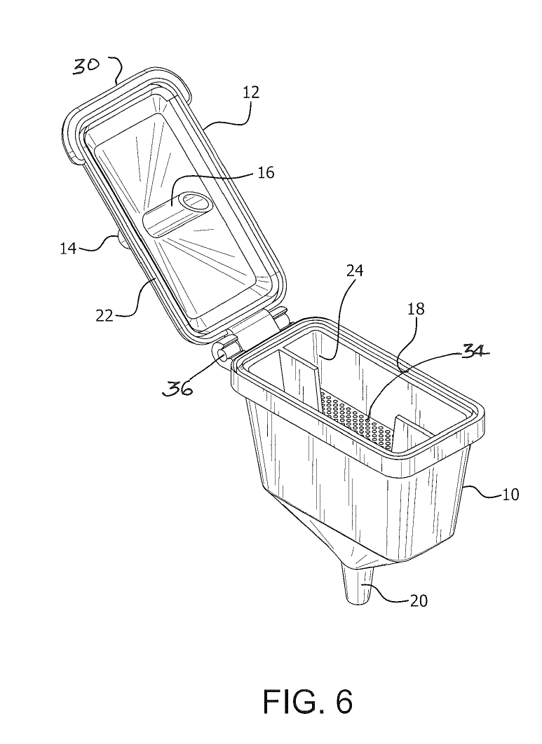

### DETAILED DESCRIPTION OF THE INVENTION

During some endoscopic procedures abnormal tissue is severed and removed via suction from the body through an endoscopic channel. A polyp trap is a device known in the art that is placed in the suction line downstream from the endoscope and acts to capture removed tissue.

Trap suction is created when the top 12 and bottom 10 of the trap are closed. A seal is created by a series of interlocking ridges 18, 22 designed into the upper 12 and lower 10 bodies of the trap that lock together to form a seal. A specimen port 14 and port extension 16 extend through the trap top 12, and a vacuum port 20 extends through the lower end of the trap body 10. There is no twisting or turning needed to open or close the trap. The trap is hinged at connection 36 like a clam shell so that the matching halves are always aligned providing ease of operation for the technician or physician assistant. Just bring the top to mate with the bottom, basket 24 in place and suction on, and the trap is ready for collection. A large flange handle 30 is designed into the upper half of the trap to assist in the easy opening of the trap against the closing pressure created by the suction. The trap is designed as an inline device with two separate sites that are capable of collecting samples, the basket 24 and the screen 26. This double collection or fail safe is unique to present trap. The inline design is more natural to the flow of vacuum lines in a typical office.

As clearly illustrated in the drawings, the basket 24 has a bottom wall 32 with a plurality of small drainage openings 34 therein. Also, the screen 26 rests at the bottom portion of the trap body 10 directly above the vacuum port 20.

Additionally, the hinge connection 36 includes a stop 38 which limits the extent of opening of the polyp trap and thereby prevents over opening and possible contamination of adjacent working surfaces.

1. A polyp trap comprising a body portion and a top portion sealingly and releasably covering the body portion, a suction entrance passageway in the top portion constructed and arranged to receive liquid and tissue during examination procedures, a removable tissue collection basket in the body portion having a bottom wall with a plurality of small openings therein constructed and arranged to collect tissue and drain liquid as liquid and tissue pass into the collection basket from the suction entrance passageway, a removable screen in the body portion below the collection basket, and a suction discharge passageway in the body portion below the movable screen connected to a suction source for the passage of liquid from the body portion.

2. A polyp trap as in claim 1 including a hinge connection between the top and bottom portions for opening and losing the top portion over the body portion about the hinge connection.

3. A polyp trap as in claim 2 including a stop at the hinge connection for limiting the extent of opening of the top portion away from the body portion.

4. A polyp trap as in claim 1 wherein both the top and bottom portions include cooperating vacuum sealing ridges where the top portion contacts the body portion upon closure whereby the top and body portions are releasably vacuum sealed together upon closure.

5. A polyp trap as in claim 1 wherein the top portion includes a handle in the form of an outwardly extending flange for moving the top portion over and away from the body portion.

6. A polyp trap as in claim 1 wherein the body portion is downwardly and inwardly tapered.
